(19) United States
(12) Patent Application Publication
(10) Pub. No.: US 2026/0096810 A1
(43) Pub. Date: Apr. 9, 2026

(54) DEVICES AND METHODS FOR BONE FIXATION AUGMENTATION

(71) Applicant: DePuy Synthes Products, Inc., Raynham, MA (US)

(72) Inventor: Todd J. KENT, Raynham, MA (US)

(73) Assignee: DePuy Synthes Products, Inc., Raynham, MA (US)

(21) Appl. No.: 18/907,001

(22) Filed: Oct. 4, 2024

(51) Int. Cl. A61B 17/00; A61B 17/68

(52) U.S. Cl. CPC A61B 17/00; A61B 2017/681

(57) ABSTRACT

An example method is provided for the approximation of two bones. The method can include engaging a first anchor with a first bone, fixating a second anchor to a second bone, and fixating a flexible segment between the first anchor and the second anchor. The flexible segment can be configured to adjust a distance between the first and second bones, and can comprise one or more suture ends extending proximally from the second anchor and the second bone. The method can further include fixating at least a first suture end of the one or more suture ends to the first bone at a first distance from the first anchor.

### FIELD

The present invention generally relates to devices and methods for bone fixation augmentation. More specifically, certain embodiments relate to systems and methods for fixation augmentation of the distal tibia and distal fibula following an injury to the corresponding syndesmotic joint.

### BACKGROUND

A syndesmotic injury results when a traumatic injury damages the ligaments that span the gap between the distal tibia and fibula. This can be the result, for example, of a high ankle sprain, with no fracture of the fibula, or, for example, can also accompany a fibular fracture in a Weber B or Weber C fracture.

A surgeon can determine the presence of a syndesmotic injury by direct visualization of the joint or through radiographic imaging while positioning the ankle in a mortise view orientation. In either case, loads are applied to the joint in either a direct lateral load applied to the fibula or by applying an external rotation load to the foot. While the load is being applied, the relative distance between the fibula and the tibia, the fibula and the talus, and the tibia and the talus are observed to determine the level of damage sustained by the ligaments that typically hold the syndesmotic joint together.

If a syndesmotic injury is found to be present, the typical treatment involves stabilizing the fibula and tibia with respect to each other in the proper orientation and holding them there throughout the soft tissue healing period to allow the ligaments to re-attach and heal. In the event of a syndesmotic injury with a corresponding fibula fracture, this is done while also stabilizing the fibular fracture, which is usually accomplished with a small fracture plate on the lateral side of the fibula.

Traditional methods of stabilization include engaging a respective anchor with the tibia and fibula bones, and connecting a flexible internal segment between the respective anchors such that the flexible segment is disposed across the syndesmosis. In some systems, the anchor engaged with the tibia bone may be inserted (e.g., screwed) into the tibia bone with the tip of the screw being in the middle of the tibia or in the medial cortex of the tibia. In other systems, the tibia anchor may instead lie on the medial side of the tibia, requiring a through or bore hole through the medial wall of the tibia. These traditional methods of stabilization can present challenges in providing sufficient enough stabilization for proper and efficient recovery.

Accordingly, alternative apparatus and methods for providing bone fixation augmentation of the distal tibia and fibula following a syndesmotic injury would be useful.

### SUMMARY

The present invention is directed to apparatus and methods for stabilizing a joint between two bones, e.g., the tibia and fibula, during the soft tissue healing period following a traumatic injury.

In an exemplary application, the apparatus and methods herein may be configured to provide substantially rigid tensile fixation between the tibia and fibula while allowing the small amount of shear and rotational motion required for a standard gait. This may make it possible for patients to return to weight-bearing earlier, which may improve clinical outcomes, and/or may also reduce the number of follow-up hardware-removal surgeries.

An example method is provided for the approximation of two bones. The method can include engaging a first anchor with a first bone, fixating a second anchor to a second bone, and fixating a flexible segment between the first anchor and the second anchor. The flexible segment can be configured to adjust a distance between the first and second bones, and can comprise one or more suture ends extending proximally from the second anchor and the second bone. The method can further include fixating at least a first suture end of the one or more suture ends to the first bone at a first distance from the first anchor.

Another example method is provided for the approximation of two bones. The method can include engaging a first anchor with a first bone, fixating a second anchor to a second bone, and fixating a flexible segment between the first anchor and the second anchor. The flexible segment can be configured to adjust a distance between the first and second bones, and can comprise one or more suture ends extending proximally from the second anchor and the second bone. The method can further include engaging the one or more suture ends with one or more fixation devices, and fixating the one or more fixation devices to the first bone at a first distance from the first anchor.

Another example method is provided for the approximation of two bones. The method can include engaging a first anchor with a first bone, fixating a second anchor to a second bone, and fixating a flexible segment between the first anchor and the second anchor. The flexible segment can include one or more suture ends extending proximally from the second anchor and the second bone. The method can further include engaging the one or more suture ends with a first fixation device, and fixating the first fixation device to an anterior side of the first bone at a first distance from the first anchor.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an example system for bone fixation augmentation according to aspects of the present invention.

FIG. 2 is an illustration of an example system for bone fixation augmentation according to aspects of the present invention.

FIG. 3A is an illustration of an example system for bone fixation augmentation according to aspects of the present invention.

FIG. 3B is an illustration of an example system for bone fixation augmentation according to aspects of the present invention.

FIG. 3C is an illustration of an example system for bone fixation augmentation according to aspects of the present invention.

FIG. 4 is an illustration of an example system for bone fixation augmentation according to aspects of the present invention.

FIG. 5 is a flowchart of an exemplary method for bone fixation augmentation according to aspects of the present invention.

### DETAILED DESCRIPTION

The example devices and methods of treatment described herein generally involve providing bone fixation augmentation of two bones, such as the tibia and fibula bones, by implanting an apparatus, including a first anchor, a second anchor, and a flexible segment, through the fibula bone and/or tibia bones, and augmenting such fixation with one or more additional fixation devices implanted in the tibia bone. The disclosed example devices and methods of treatment thus aid in restricting rotation of the fibula bone with respect to the tibia bone, helping to improve patient recovery.

Various example systems and methods are presented herein. Features from each example are combinable with other examples as understood by persons skilled in the pertinent art.

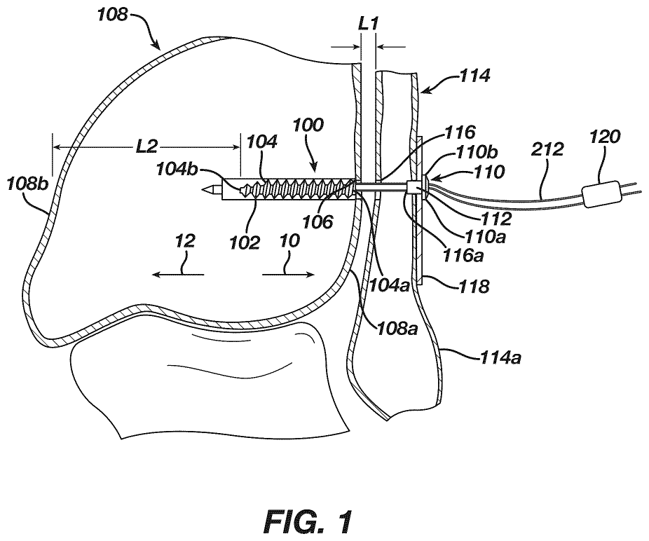

FIG. 1 is an illustration of an example system 100 for bone fixation augmentation. The system 100 includes an apparatus 102 for the approximation of two bones, such as the tibia and fibula bones. The apparatus 102 generally includes a first anchor 104, which includes a proximal end 104a and a distal end 104b configured for insertion into a first hole 106 in a first bone 108, e.g., a tibia; a second anchor 110, which includes a proximal end 110a and a distal end 110b configured for insertion into a second hole 112 in a second bone 114, e.g., a fibula; and a flexible segment 116 extending between the first anchor 104 and the second anchor 110.

The flexible segment 116 is configured to adjust a distance L1 between the first and second bones 108, 114, and the second anchor 110 releasably engages the flexible segment 116 such that the distance L1 can be adjusted and fixed between the first and second bones 108, 114. The flexible segment 116 includes one or more suture ends 212 that extend proximally 10 from the second anchor 110 and the second bone 114. One or more of the suture end(s) 212 can be engaged with (e.g., fixated to) a fixation device 120 that can be fixated to the first bone 108 for augmenting the bone fixation of system 100, as further discussed below.

The first anchor 104 can include any type of suture anchor, and can be manufactured from a surgical stainless steel or other suitable biocompatible material, such as 316 LVM stainless steel, titanium, and other suitable materials, such as nitinol, bio-absorbables, or non-absorbables (e.g., PEEK). First anchor 104 can also include an “all-textile” anchor (e.g., VERSALOOP™). The first anchor 104 can pass through the second hole 112 in the second bone 114 and be inserted (e.g., screwed, threaded, or pushed) within the first bone 108 in the first hole 106. The first anchor 104 can be inserted into the first bone 108 from its distal end 104b to its proximal end 104a such that the first anchor 104 does not extend all the way through the first bone 108. That is, the first hole 106 is disposed on a first side 108a (e.g., the lateral or proximal 10 side) of the first bone 108, and the first anchor 104 is configured such that a distance L2 remains between the distal end 104b of the first anchor 104 and a second side 108b (e.g., the medial or distal 12 side) of the first bone 108. In this way, no through or bore hole is required to be made in the second side 108b of the first bone 108.

The flexible segment 116 can be manufactured out of a variety of fibers or filaments including but not limited to polymer filaments (e.g., HMWPE, UHMWPE, PET, PTFE, PEEK, PEKK, PLA, PLLA, etc.), metallic filaments (e.g., Nitinol, Titanium, Titanium alloys, Tantalum, Stainless Steel, etc.), or organic filaments (e.g., Collagen, Silk, etc.), or other filaments such as carbon fiber or carbon nanotubes, etc., and can be made of resorbable and/or biologic materials. Flexible segment 116 can include, but is not limited to, a coreless suture, a suture with a jacket and a central core, a tape, or any other tension member available or contemplated, can be poly-coated or uncoated, and can include collagen. Flexible segment 116 can extend between the distal end 110b of the second anchor 110 and the proximal end 104a of the first anchor 104 to adjust the distance L1 between the first and second bones 108, 114. Additionally, the flexible segment 116 can extend beyond the distal end 110b of the second anchor 110 (e.g., suture threads 212 can be manipulated proximally to the second bone 114).

The second anchor 110 can be manufactured from a surgical stainless steel or other suitable biocompatible material, such as 316 LVM stainless steel, titanium, other suitable materials, such as nitinol, bio-absorbables, or non-absorbables (e.g., PEEK). Second anchor 110 can also include an “all-textile” anchor (e.g., VERSALOOP™). Second anchor 110 can be coupled to a bone plate 118 which itself can be coupled to a first side 114a of the second bone 114. As those having skill in the pertinent art will appreciate, bone plate 118 may include one or more holes through which a physician may deliver the apparatus 102 for approximation of the first and second bones 108, 114, as further described below.

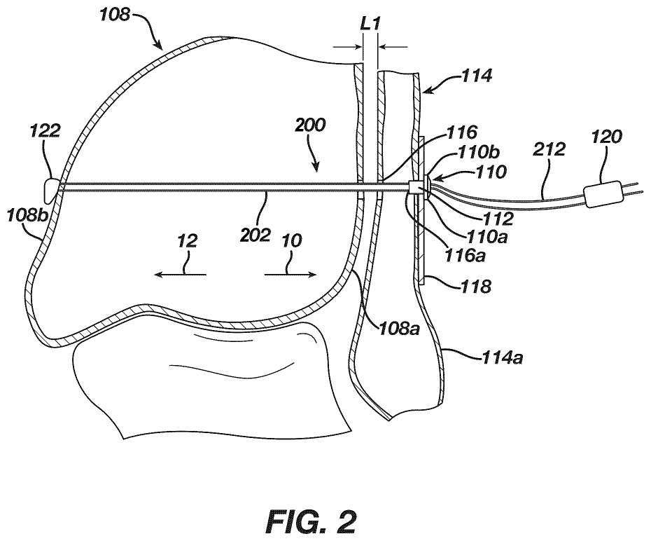

FIG. 2 is an illustration of another example system 200 for bone fixation augmentation. The system 200 includes an apparatus 202 for the approximation of two bones, such as the tibia and fibula bones. Apparatus 202 may have one or more of the same or similar features as those described above with respect to apparatus 102 of system 100. However, rather than apparatus 202 include the first anchor 104 fixed in the first bone 108, apparatus 202 instead includes a locking mechanism 122 configured to engage with the first bone 108 by lying against a second side 108b (e.g., the medial or distal side) of the first bone 108. In such system, the flexible segment 116 extends between the locking mechanism 122 on the medial or distal side of the first bone 108 and the second anchor 110 fixed in the second bone 114. Similar to apparatus 102, apparatus 202 includes fixation device 120 and suture end(s) 212 of flexible segment 116.

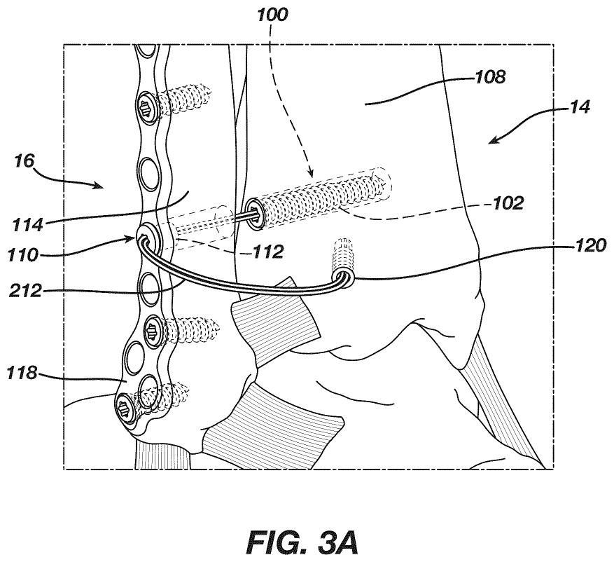

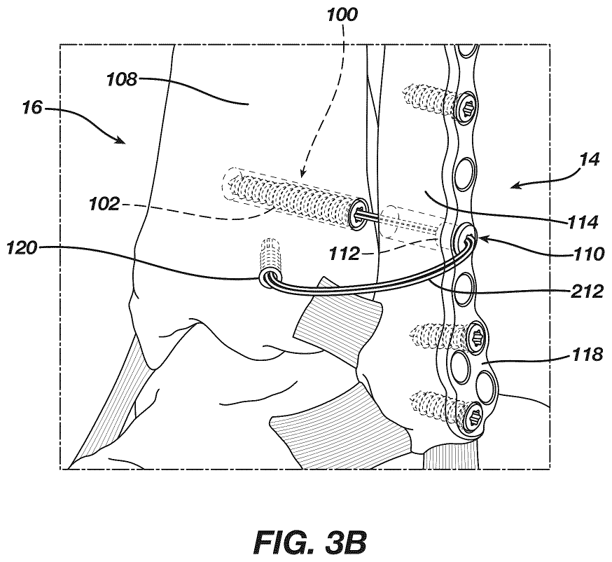

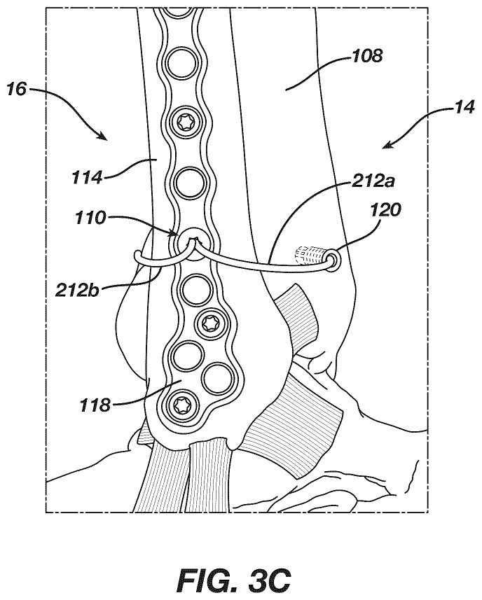

FIGS. 3A-3C are illustrations of various methods for augmenting the fixation systems discussed herein by implanting the suture ends 212 of the flexible segment 116 into the first bone 108 via the fixation device 120. As shown in FIG. 3A, the suture end(s) 212 can be engaged with or fixed to fixation device 120 (e.g., a knotless anchor) that can then be fixed or implanted into the posterior side 14 of the first bone 108. FIG. 3B provides an alternative augmentation approach whereby the fixation device 120 can be fixed or implanted into the anterior side 16 of the first bone 108. FIG. 3C provides yet another alternative augmentation approach whereby two fixation devices 120a, 120b can be respectively fixed or implanted into the posterior side 14 and anterior side 16 of the first bone 108. A benefit of such fixation augmentation is it helps restrict rotation of the second bone 114 with respect to the first bone 108.

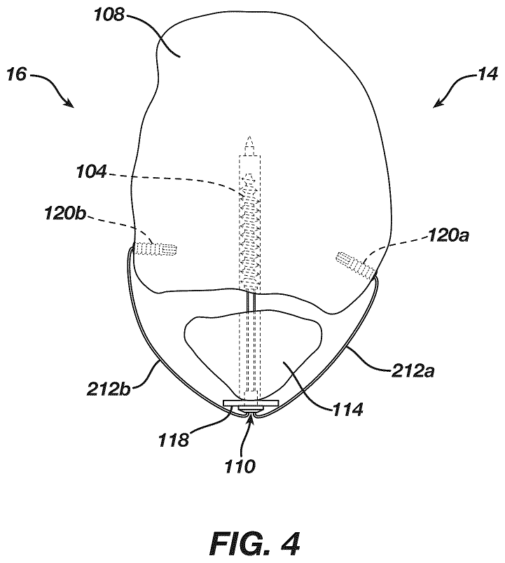

FIG. 4 is a birds-eye view of the example system 100 shown in FIG. 1. That is, an overhead perspective of the first and second bones 108, 114 is shown, with apparatus 102 engaged with the first and second bones 108, 114 as discussed above. As in FIG. 3C, FIG. 4 illustrates how two fixation devices 120a, 120b can be used for providing fixation augmentation of the first and second bones 108, 114. That is, one or more suture end(s) 212a of the suture end(s) 212 may be engaged with a first fixation device 120a, which itself may be fixed or implanted in the posterior side 14 of the first bone 108. Fixation device 120a may be fixed or implanted such that a first distance D1 remains between the fixation device 120a and the first anchor 104 inserted into the first bone 108, as discussed above. Additionally, one or more suture end(s) 212b of the suture end(s) 212 may be engaged with a second fixation device 120b, which itself may be fixed or implanted in the anterior side 16 of the first bone 108. Fixation device 120b may be fixed or implanted such that a second distance D2 remains between the fixation device 120b and the first anchor 104 inserted into the first bone 108, as discussed above. First and second distances D1, D2 may be of equal distance, or may be different distances with respect to one another. While FIG. 4 illustrates a scenario in which both first and second fixation devices 120a, 120b are used, it should be appreciated that either first or second fixation device 120a, 120b could alternatively be used on its own, as illustrated in FIGS. 3A and 3B. In any of these alternative fixation augmentation systems, it should also be understood that a single suture end and/or multiple suture ends can be engaged with one or more fixation devices 120 being fixated into the first bone 108.

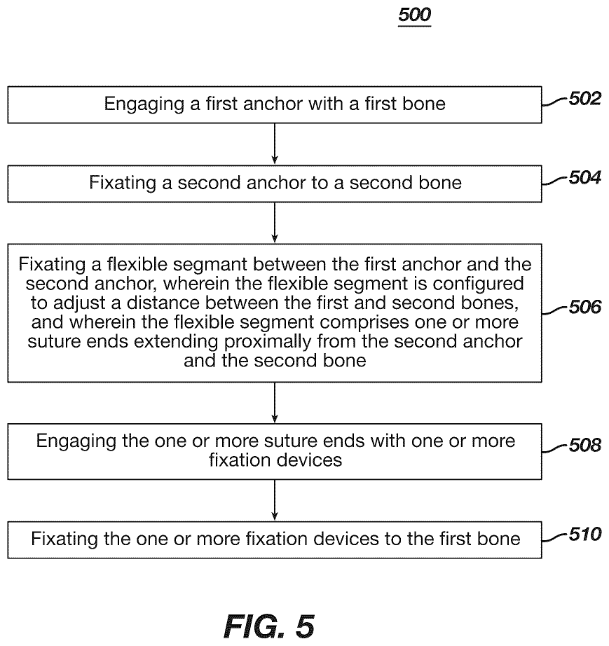

FIG. 5 is a flowchart of a method 500 for the approximation of two bones.

In block 502, the method can include engaging a first anchor (e.g., first anchor 104 or 122) with a first bone (e.g., first bone 108). As discussed herein, first anchor 104 can be used by inserting it into the first bone 108, while first anchor 122 can be used by engaging first anchor 122 with the medial side of the first bone 108.

In block 504, the method can include fixating a second anchor (e.g., second anchor 110) to a second bone (e.g., second bone 114). As discussed herein, the second anchor may be inserted into the second bone via a nail (e.g., nail 118).

In block 506, the method can include fixating a flexible segment (e.g., flexible segment 116) between the first anchor and the second anchor. As discussed herein, depending on which first anchor is being used, the flexible segment may extend only into the first bone, or may extend beyond the medial wall of the first bone. In either case, however, the flexible segment is configured to adjust a distance between the first and second bones, and can include suture end(s) (e.g., suture end(s) 212) that extend proximally beyond the second anchor and second bone.

In block 508, the method can include engaging the suture end(s) with one or more fixation device(s) (e.g., fixation devices 120a, 120b), such as a knotless anchor. The suture end(s) can be looped through the fixation device 120 or connected to fixation device 120 using a variety of methods.

In block 510, the method can include fixating the fixation device(s) to the first bone. As discussed above with respect to FIG. 4, the fixation device(s) can be fixed in the first bone such that a distance (e.g., D1 or D2) remains between the fixation device(s) and the first anchor. As discussed herein, the one or more fixation devices can be inserted into or fixed in the anterior and/or posterior sides of the first bone, helping to restrict rotation of the second bone with respect to the first bone.

In some examples, disclosed systems or methods may involve one or more of the following clauses:

Clause 1: A method for the approximation of two bones, the method comprising: engaging a first anchor with a first bone; fixating a second anchor to a second bone; fixating a flexible segment between the first anchor and the second anchor, wherein the flexible segment is configured to adjust a distance between the first and second bones, and wherein the flexible segment comprises one or more suture ends extending proximally from the second anchor and the second bone; and fixating at least a first suture end of the one or more suture ends to the first bone at a first distance from the first anchor.

Clause 2: The method of clause 1, wherein engaging the first anchor with the first bone comprises fixating the first anchor to the first bone.

Clause 3: The method of clause 2, wherein fixating the first anchor to the first bone comprises inserting a distal end of the first anchor into a first hole in a first side of the first bone such that there is a first distance between the distal end of the first anchor and a second side of the first bone.

Clause 4: The method of clause 1, wherein engaging the first anchor with the first bone comprises engaging the first anchor with a first side of the first bone.

Clause 5: The method of clause 1, wherein fixating at least the first suture end of the one or more suture ends to the first bone comprises: engaging the first suture end with a fixation device; and fixating the fixation device to the first bone.

Clause 6: The method of clause 5, wherein fixating the fixation device to the first bone comprises fixating the fixation device to a posterior side of the first bone.

Clause 7: The method of clause 5, wherein fixating the fixation device to the first bone comprises fixating the fixation device to an anterior side of the first bone.

Clause 8: The method of clause 5, wherein fixating at least the first suture end of the one or more suture ends to the first bone comprises: engaging the first suture end with a first fixation device; fixating the first fixation device to a posterior side of the first bone; engaging a second suture end with a second fixation device; and fixating the second fixation device to an anterior side of the first bone.

Clause 9: The method of clause 5, wherein the fixation device comprises a knotless anchor.

Clause 10: The method of clause 5, wherein fixating at least the first suture end of the one or more suture ends to the first bone restricts rotation of the second bone with respect to the first bone.

Clause 11: A method for the approximation of two bones, the method comprising: engaging a first anchor with a first bone; fixating a second anchor to a second bone; fixating a flexible segment between the first anchor and the second anchor, wherein the flexible segment is configured to adjust a distance between the first and second bones, and wherein the flexible segment comprises one or more suture ends extending proximally from the second anchor and the second bone; engaging the one or more suture ends with one or more fixation devices; and fixating the one or more fixation devices to the first bone at a first distance from the first anchor.

Clause 12: The method of clause 11, wherein fixating the one or more fixation devices to the first bone comprises fixating the one or more fixation devices to a posterior side of the first bone.

Clause 13: The method of clause 11, wherein fixating the one or more fixation devices to the first bone comprises fixating the one or more fixation devices to an anterior side of the first bone.

Clause 14: The method of clause 11, wherein: engaging the one or more suture ends with the one or more fixation devices comprises: engaging a first suture end of the one or more suture ends with a first fixation device of the one or more fixation devices; and engaging a second suture end of the one or more suture ends with a second fixation device of the one or more fixation devices; and fixating the one or more fixation devices to the first bone comprises: fixating the first fixation device to a posterior side of the first bone; and fixating the second fixation device to an anterior side of the first bone.

Clause 15: The method of clause 11, wherein the one or more fixation devices comprise a knotless anchor.

Clause 16: The method of clause 11, wherein fixating the one or more fixation devices to the first bone restricts rotation of the second bone with respect to the first bone.

Clause 17: A method for the approximation of two bones, the method comprising: engaging a first anchor with a first bone; fixating a second anchor to a second bone; fixating a flexible segment between the first anchor and the second anchor, wherein the flexible segment comprises one or more suture ends extending proximally from the second anchor and the second bone; engaging the one or more suture ends with a first fixation device; and fixating the first fixation device to an anterior side of the first bone at a first distance from the first anchor.

Clause 18: The method of clause 17, wherein engaging the first anchor with the first bone comprises inserting a distal end of the first anchor into a first hole in a first side of the first bone such that there is a first distance between the distal end of the first anchor and a second side of the first bone.

Clause 19: The method of clause 17, wherein engaging the first anchor with the first bone comprises engaging the first anchor with a first side of the first bone.

Clause 20: The method of clause 17, wherein fixating the first fixation device to the anterior side of the first bone restricts rotation of the second bone with respect to the first bone.

The descriptions contained herein are examples of embodiments of the invention and are not intended in any way to limit the scope of the invention. As described herein, the invention contemplates many variations and modifications of structures and methods, including alternative materials, alternative configurations of component parts, and alternative method steps. Modifications and variations apparent to those having skill in the pertinent art according to the teachings of this disclosure are intended to be within the scope of the claims which follow.

What is claimed is:

1. A method for the approximation of two bones, the method comprising:
engaging a first anchor with a first bone;
fixating a second anchor to a second bone;
fixating a flexible segment between the first anchor and the second anchor,
wherein the flexible segment is configured to adjust a distance between the first and second bones, and
wherein the flexible segment comprises one or more suture ends extending proximally from the second anchor and the second bone; and
fixating at least a first suture end of the one or more suture ends to the first bone at a first distance from the first anchor.

2. The method of claim 1, wherein engaging the first anchor with the first bone comprises fixating the first anchor to the first bone.

3. The method of claim 2, wherein fixating the first anchor to the first bone comprises inserting a distal end of the first anchor into a first hole in a first side of the first bone such that there is a first distance between the distal end of the first anchor and a second side of the first bone.

4. The method of claim 1, wherein engaging the first anchor with the first bone comprises engaging the first anchor with a first side of the first bone.

5. The method of claim 1, wherein fixating at least the first suture end of the one or more suture ends to the first bone comprises:
engaging the first suture end with a fixation device; and
fixating the fixation device to the first bone.

6. The method of claim 5, wherein fixating the fixation device to the first bone comprises fixating the fixation device to a posterior side of the first bone.

7. The method of claim 5, wherein fixating the fixation device to the first bone comprises fixating the fixation device to an anterior side of the first bone.

8. The method of claim 5, wherein fixating at least the first suture end of the one or more suture ends to the first bone comprises:
engaging the first suture end with a first fixation device;
fixating the first fixation device to a posterior side of the first bone;
engaging a second suture end with a second fixation device; and
fixating the second fixation device to an anterior side of the first bone.

9. The method of claim 5, wherein the fixation device comprises a knotless anchor.

10. The method of claim 5, wherein fixating at least the first suture end of the one or more suture ends to the first bone restricts rotation of the second bone with respect to the first bone.

11. A method for the approximation of two bones, the method comprising:
engaging a first anchor with a first bone;
fixating a second anchor to a second bone;
fixating a flexible segment between the first anchor and the second anchor,
wherein the flexible segment is configured to adjust a distance between the first and second bones, and
wherein the flexible segment comprises one or more suture ends extending proximally from the second anchor and the second bone;
engaging the one or more suture ends with one or more fixation devices; and
fixating the one or more fixation devices to the first bone at a first distance from the first anchor.

12. The method of claim 11, wherein fixating the one or more fixation devices to the first bone comprises fixating the one or more fixation devices to a posterior side of the first bone.

13. The method of claim 11, wherein fixating the one or more fixation devices to the first bone comprises fixating the one or more fixation devices to an anterior side of the first bone.

14. The method of claim 11, wherein:
engaging the one or more suture ends with the one or more fixation devices comprises:
engaging a first suture end of the one or more suture ends with a first fixation device of the one or more fixation devices; and
engaging a second suture end of the one or more suture ends with a second fixation device of the one or more fixation devices; and
fixating the one or more fixation devices to the first bone comprises:
fixating the first fixation device to a posterior side of the first bone; and
fixating the second fixation device to an anterior side of the first bone.

15. The method of claim 11, wherein the one or more fixation devices comprise a knotless anchor.

16. The method of claim 11, wherein fixating the one or more fixation devices to the first bone restricts rotation of the second bone with respect to the first bone.

17. A method for the approximation of two bones, the method comprising:
engaging a first anchor with a first bone;
fixating a second anchor to a second bone;
fixating a flexible segment between the first anchor and the second anchor,
wherein the flexible segment comprises one or more suture ends extending proximally from the second anchor and the second bone;
engaging the one or more suture ends with a first fixation device; and
fixating the first fixation device to an anterior side of the first bone at a first distance from the first anchor.

18. The method of claim 17, wherein engaging the first anchor with the first bone comprises inserting a distal end of the first anchor into a first hole in a first side of the first bone such that there is a first distance between the distal end of the first anchor and a second side of the first bone.

19. The method of claim 17, wherein engaging the first anchor with the first bone comprises engaging the first anchor with a first side of the first bone.

20. The method of claim 17, wherein fixating the first fixation device to the anterior side of the first bone restricts rotation of the second bone with respect to the first bone.
